(19) United States
(12) Patent Application Publication
(10) Pub. No.: US 2014/0329324 A1
(43) Pub. Date: Nov. 6, 2014

(54) FUNCTIONAL UNLIMITED CULTURE MEDIUM HAVING A NATURAL BIO-COMPOSITE INDUCER

(71) Applicant: Universidad de Antioquia, Medellin (CO)

(72) Inventors: Amanda Ines Mejia Gallon, Medellin (CO); Carolina Arboleda Echavarria, Medellin (CO)

(73) Assignee: Universidad de Antioquia, Medellin (CO)

(21) Appl. No.: 14/360,477

(22) PCT Filed: Nov. 22, 2012
(86) PCT No.: PCT/IB12/56642

§ 371 Date: May 23, 2014

(30) Foreign Application Priority Data
Nov. 23, 2011 (CO) 11160657

(51) Int. Cl. C12N 5/00

(52) U.S. Cl. CPC C12N 5/0025; C12N 2500/30
USPC 435/420; 435/431

(57) ABSTRACT

A culture medium with natural inducer for biocomposites. In a general aspect the present disclosure is an indefinite functional culture medium with a natural inducer for biocomposites comprising: i) biomass plant incorporating high copper content, vitamins and trace minerals; and ii) a natural inducer, that is vegetable vinegar.

### CLAIM TO PRIORITY

The present application is a continuation of, and claims the benefit of, PCT/IB2012/056642 (PCT Published Application WO2013/076686), filed as an international application on Nov. 22, 2012; the international application claims the benefit of Colombian national application 11-160657, filed Nov. 23, 2011. Both of these applications are incorporated by reference in their entirety.

### FIELD

The present disclosure relates to the field of biotechnology, specifically to cell cultures. More specifically it relates to development of functional and sustainable culture media that optimize production of biocomposites

### BACKGROUND

Cell culture is the process by which prokaryotic or eukaryotic cells are grown under controlled conditions. In practice the term “cell culture” is normally used in reference to the cultivation of isolated cells. Currently, cell culture is a routine laboratory technique in biotechnology research. Cells are grown and maintained at an appropriate temperature and gas mixture in an incubator. Culture conditions vary widely for each cell type, and variation of conditions for a particular cell type may lead to the expression of various phenotypes.

Besides temperature and gas mixture, the most commonly varied factor in culture systems is the growth medium or culture. These growth media or cultures are liquid, semi-solid or solid nutrients on which the cells are cultivated for multiplication and growth. In the most general division of culture media differentiation is made between those designed for growth of microorganisms and growth of animal and plant cells. The most common media for microorganisms are the broth and agar plates. The difference between the media for cell cultures and microorganisms is that the media for eukaryotic cells must be supplemented with substances such as hormones or growth factors.

Another important distinction between types of culture media is that they can be definite or indefinite. A definite medium (also known as synthetic or chemically defined medium) is a medium in which all chemicals used are known and it does not contain yeast or animal or plant tissues. Defined media provide cultured cells with trace elements, vitamins and a carbon and nitrogen source, where the sugars are the most used source for carbon, and ammonium salts or nitrates are the nitrogen source.

Indefinite media (also known as basal or complex media) are media containing: a carbon source, a variety of water, and salts, in combination with complex ingredients, which consist of a mixture of many chemical species in unknown proportions, such as yeast extract and casein hydrolyzate. Indefinite means are selected many times for the nutritional needs of organisms or even designed for agencies that have not been grown in defined media.

In another important classification culture media are classified according to the origin of the components that comprise it. Natural culture media are prepared from natural substances of plant or animal origin, specifically tissue extracts or infusions, whose chemical composition is not exactly known. Synthetic media, however, correspond with culture media of qualitatively and quantitatively defined chemical composition and are used to obtain reproducible results. For their use in practice culture or growth media are also classified into minimal media and differential media.

A minimal medium is one which contains the minimum possible nutrients for cell growth, generally without the presence of amino acids. Typically, these media contain: a carbon source, various salts, which allow the synthesis of proteins and nucleic acids, and water. Differential media are those that include some form of added indicator which allows identification of chemical reactions that occur during cell growth. There are also supplemented minimal media which are a type of minimal medium that also contains a selected agent, usually amino acids and sugars, as a supplement. This supplementation allows the cultivation of specific lines of autotrophic organisms. These supplemented or enriched media contain the nutrients required to support growth of a wide variety of organisms. Other media are media selectively used for the growth of selected organisms and allow differentiation between various organisms. There are even transport culture media which maintain the viability of the organisms during storage, containing only salts and buffer solutions and lacking sources of carbon, organic nitrogen and growth factors.

In the context of this disclosure are also defined functional media which by design and initial content of natural elements are an excellent substrate for cell growth, especially for microorganisms, fungi and yeasts. These operating media are designed to reproduce under natural physiological conditions, and are sustainable and affordable. All culture media or growth, regardless of their type, contain different nutrients ranging from simple sugars to complex substances such as protein extracts.

The design recipes for culture media and growth conditions optimization is done in most cases to potentiate the primary and secondary metabolism, this being the subject of much research in the area of biotechnology. Within the design of these recipes modifying sources of carbon, nitrogen, minerals, vitamins and adding growth inducers is one of the leading practices for optimizing culture conditions and for obtaining the desired products of primary and secondary metabolism in culture cells.

Among the carbon sources most commonly used for the design of culture media are glucose, sucrose, lactose, hydrocarbons, fats present in starch, cane sugar, and milk, and as a nitrogen source mainly proteins, ammonium nitrate, and nitrogen present in ammonium, pure ammonium nitrate salt, air and soy protein (Thome Vieira et al., 2008; Atalla et al., 2008). Particularly, some of the most used ingredients as input of carbon and nitrogen in culture media are agar, glucose, peptone, casein, malt extract, and yeast extract (De Corzo et al., 2009, Martinko et al., 1998, Davis et al., 1990). More particularly, patent document US2009/0005340 relates to a method for culturing yeast cells in liquid medium, preferably malt extract and peptone, as carbon source, for intracellular or extracellular production of one or more bioactive agents between 7 and 24 days. In turn, patent document CN1935992 discloses an inexpensive medium composed of agricultural products, for a clean and highly efficient production of a secondary metabolite. Patent document US2009/0005340 also discloses a liquid culture medium under conditions which result in the production of bioactive compounds, specifically pharmaceutically active ones.

Alternatively, within the design of the culture media are also included additives that may act as potential inducers of biocomposites of interest. These inducers can even direct the synthesis of a product among several that are generally produced. Generally, these additives are used as a supplement to the basal medium after several days of cultivation in order to not inhibit cell growth. It is common to find media supplemented from the initial recipe because these inducers can inhibit cell growth. The addition of these inducers also seeks to optimize the culture conditions, and reduce growing time and costs associated with it.

In its broadest definition the inducers used for addition or supplementation of culture media can be chemical or natural. The addition or supplementation of culture media with chemicals inducers as compounds of interest is reported for all cell types. Further investigations showed the influence of metals on the growth phase in bacterial cells (Billard et al., 2001). Holker and others published on improved expression of manganese peroxidase and laccase in the presence of manganese and aromatics from white rot wood fungi (Holker et. al., 2000). Merroun also described the interaction of metals and bacteria in bioremediation reactions (Merroun et. al, 2007). Kuryshe and others also demonstrated that iron overload increases the release of arachidonic acid and its incorporation in phosphatidylcholine, and the induction of cyclooxygenase-2 and the production of eicosanoids, in neonatal rat ventricular myocytes (Kuryshe et. al. 2001). Legorreta et al. also demonstrated the increased synthesis of thiols in seagrass tissues in response to cadmium exposure (Legorreta et. al., 2001). Specifically, phenolic compounds, flavonoids, metal ions, alkaloids, stilbenes, essential oils and fatty acids have been used as chemical inducers, among others (Roat et al., 2009; Ramani et al., 2009; Maharik et al., 2009).

More specifically, among the most used chemical inducers are phenylacetic acid, substituted benzoic acids and some amino acids specific for antibiotic production; ABTS (2,2′-azino-bis-(3-ethylbenzothiazoline-6-sulfonic acid), metal ions such as Mn+2 and veratryl alcohol to produce enzymes (Bourbonnais et al, 1995; Johannes et al., 1996; Majcherczyk et al., 1999); ferulic acid, aromatic amino acids, isoeugenol for synthesis of flavors (Bartholomew et al., 1997, Ranadive et al, 1994, Dignum et al, 2001; Havkin-Frenkel et. al., 2005). It has even been proven that different aromatic compounds can increase the quantities of metabolites of interest produced (Collins et al, 1997, Chen, et al, 2003). In 1985 Faison and Kirk specifically demonstrated that aromatic compounds such as veratryl alcohol and substrates such as manganese, activated production of enzymatic metabolites to be used as supplements for minimum synthetic media.

Experimental evidence exists for the addition of compounds such as xylidine, ferulic acid, veratryl alcohol and pyrogallol as inducers of compounds of interest (Minusi et. al., 2007). Particularly, the use synthetic or even natural media has been reported supplemented with aromatic compounds such as trinitrotoluene, xylidine, catechol, pyrogallol and others (Elisashvili et. al., 2010). Other compounds such as gallic acid, vanillic acid, guaiacol, and others have also been reported as enhancers of enzymatic production and mediators in cellular functions (Niladevi et. al., 2008). However, it has been demonstrated that substances based on phenol (for example, phenol, guaiacol, tannic acid, eugenol) undergo conversion and produce products that may be harmful to the skin (and possibly to the airways). Each of these compounds have phenolic hydroxyl groups that are easily oxidized to produce reactive compounds such as quinones (Buckley et. al., 2006).

Specifically, we evaluated the acute toxicity of compounds such as guaiacol in mice, associated with cancer and complex organ dysfunctions. It is therefore concluded that it is an extremely toxic substance whose use should be restricted or prohibited (Martinez et. al., 2009). For its part, Dos Santos et al. concluded in 2004 that catechol is a cytotoxic compound, being a metabolite of benzene, and probably exerts cytotoxicity due to superoxide and quinones generation in self-oxidation. Also, the widespread carcinogenic effect of compounds such as xylidine has been shown in animal models (National Toxicology Program (Cas No. 87-62-7)).

Similarly, there are reports that because of the relative inertia of nitro many nitroaromatics can be environmentally recalcitrant. 2,4,6-trinitrotoluene (TNT) is a compound that is widely used as an explosive and presents toxicity to the natural microbial flora of soil and water (Baggy et. al., 1999). The high toxicity of the chemical inducers used for supplementation of culture media is another limitation for addition in recipes for culture media with functional end-use applications in humans and animals. Alternatively to the phenolic compounds and as chemical inducers, there are also reports of supplementation research with copper and Fe³⁺ ions where ethanol and Mn²⁺ ions have been used to induce the production of metabolites (Fonseca et al, 2010; Grove et. al., 2008). In 2001 Singhal and Rathore studied the influence of zinc and copper in a synthetic basic medium for cellular growth and enzyme production of metabolites, demonstrating an increase in cellular activity (Rathore et. al., 2001).

More particularly, patent CN101638621 discloses a culture medium comprising wheat bran, sugar, baking powder, potassium dihydrogen phosphate, magnesium sulfate, vitamin B1, Cu²⁺, Zn²⁺ and Mn²⁺ in aqueous solution. As a result of this disclosure is disclosed a considerable increase in the production of secondary metabolites of interest. However, these chemical additives should be added to supplement the initial culture medium in low amounts due to their interference with cell growth and high cost. Optionally, natural inducers have been reported by their affinity to cell growth and cost reduction. Specifically, included as natural inducers in culture media are bagasse, wheat bran, rice bran, bran corn, husk rice, peel the banana, beet pulp, pulp apple, wheat flour, corn flour, steamed rice, starch, and coffee pulp, among others (Pandey et. al., 2000).

The inductive effect of chitosan on the production of secondary metabolites is also known. (Lockwood et al, 2007; Chang et al, 1998). In patent document CN1935992 the culture medium for production of secondary metabolites comprises corn flour, bran, peanut bran and water. Patent document C12N000902 discloses a clean and effective cornmeal fermentation medium for cell growth comprising bran, grains, nuts, and water.

In U.S. Pat. No. 7,883,708 a conventional culture medium was supplemented with isoflavones (soy) after several days of culture for production of biocomposites of interest. Document US20090311751 discloses a submerged fermentation in a variety of lignocellulosic substrates of organic waste, such as waste from ethanol production with barley or tangerine peels. Supplementation of culture medium with organic waste, more specifically with the products of ethanol production from barley, increases the production of metabolites. With the addition of other fruit processing residues, preferably citrus residues, as growth substrate and source of inductive components, an increase in the production of biocomposites of interest is also achieved.

Further disclosures referenced fermentation using plant derivatives as additives to the culture media. Specifically, patent document KR100949670 indicates the use of a solid medium which includes wood vinegar, various types of trees and parts thereof in up to 50% for a bacterial strain growth. Cultured material is produced by fermentation of wood vinegar in between. Patent document WO2010147345 indicates the growth of a novel bacterial strain in a culture medium containing up to 50% of pyroligneous liquid. This medium allows animal consumption of the strain grown and other industrial applications of the same. However, natural ingredients—media and inducers—usually consist of complex components that are part of agribusiness resources and are conducted under solid or semisolid conditions that have some limitations in the culture. The solid nature of the medium causes problems when measuring fermentation parameters such as pH, temperature, moisture content and the concentration of substrate and product; processes of mass transfer are limited by diffusion, and many engineering aspects such as reactor design and scaling are poorly characterized (Pandey et. al., 2009).

Furthermore, natural derivatives have limitations in their characterization and traceability. Consequently, there is still much research and development in natural, sustainable, inexpensive media that induce different phenotypes or optimize the production of biocomposites reproducibly.

### SUMMARY

Although in the prior art culture media are disclosed for the production of metabolites from fermentation cells, media reported do not include natural substrates as the sole source of carbon, nitrogen, vitamins, minerals and copper and use chemical inducers and commercial solutions of trace elements. In particular, there remains a need for natural resources including natural nontoxic inducers with high traceability for production optimization of biocomposites. The present disclosure provides an indefinite functional culture medium comprising a natural substrate as the sole source of carbon, nitrogen, vitamins, minerals and copper and a pharmaceutical grade natural inducer with surprising and reproducible effects on the production of biocomposites. The culture medium of the present disclosure contains a natural inducer of refined pharmaceutical grade as part of the initial recipe, preventing further supplementation of the medium or culturing time. The culture medium of the disclosure is sustainable and allows reducing time and cost of cell culture processes and obtaining biocomposites of interest.

The disclosed medium provides a natural environment, including inexpensive and nontoxic biomass and inducers of plant origin. The medium of this disclosure can be designed to produce multiple biocomposites. The composition of the disclosed medium provides optimal conditions for cell culture and production of biocomposites, without added toxic or synthetic substances or expensive supplements. Biocomposites obtained with the disclosed medium are achieved in a short time compared to conventional culture and in higher concentrations than those found in the prior art.

One embodiment of the disclosure is a functional unlimited culture medium having a natural biocomposite inducer. The culture medium includes a plant biomass incorporating at least 50 micromoles of copper and vitamins and trace minerals; and a natural inducer comprising pharmaceutical grade stable vegetable ligneous vinegar.

Another embodiment of the disclosure is a functional unlimited culture medium having a natural biocomposite inducer. The functional unlimited culture medium includes a plant biomass incorporating: at least 50 micromoles of copper; vitamins in a concentration of at least 10 micrograms per 100 grams; and trace minerals in a concentration of up to 10000 milligrams per 100 grams. The functional unlimited culture medium also includes a natural inducer comprising a pharmacological grade of stable vinegar from Bambusoideae.

Yet another embodiment of this disclosure is a process for culturing microorganisms by steps of cell growth carried out in the medium described herein.

The present disclosure includes these and many other embodiments, only some of which are described herein.

### BRIEF DESCRIPTION OF THE FIGURES

FIG. 1. Production of Biocomposite 1 in different eukaryotic cells -▪-Sp1, -□-Sp2. -▴-Sp3. -▾-Sp4. -♦-Sp5.

FIG. 2. Production of Biocomposite 1 in different eukaryotic cells -▪-Sp1, -□-Sp2. -▴-Sp3. -▾-Sp4. -♦-Sp5. in the presence of a medium supplemented with a Copper inducer at concentrations between 100 and 759 μM for 21 days of cultivation

FIG. 3. Chromatogram of standard Biocomposite 2

FIG. 4. Set of chromatograms of samples with different kinds of treatment, and where the changes in concentration of Biocomposite 2 are observed.

FIG. 5. Chromatogram with peaks associated with the production of Biocomposite 2 from a synthetic media environment and the disclosed media.

### DETAILED DESCRIPTION

In one aspect of the disclosure there is defined as a preferred embodiment, without limiting its scope, a culture medium including plant biomass incorporating high copper content, trace vitamins and minerals that is a mixture of cereals and others. The cereal mixture may be a mixture of maize and wheat, carob, or bamboo shoots and combinations thereof. The medium may be a cereal mixture containing an amount of copper that does not exceed millimolar concentration, vitamins in at least micromolar concentration and trace minerals and not exceeding millimolar concentration. One embodiment includes, as a source of carbon and energy, a glucose syrup of corn or cassava. Specifically, an embodiment includes a natural inducer which is a vegetable vinegar obtained by a process that includes pyrolysis, rest and fractional distillation, to obtain a refined pharmaceutical grade vinegar. Vinegar is composed of phenolic and polyphenolic compounds, organic acids, alcohols, cyclic ketones and other aromatic hydrocarbons, with trace amounts present. More specifically, the natural inducer medium of the disclosure is a refined vegetable vinegar, preferably a grass vinegar. More preferably, the natural inducer medium of the disclosure is a refined Bambusoideae vinegar in which a mixture of phenolic and polyphenolic compounds are present in trace amounts.

The medium of the disclosure, in its preferred embodiment, is plant biomass containing an inversely proportional relation to the inducer. The medium of the disclosure in its preferred embodiment is liquid or lyophilized. Optionally, the medium of the disclosure, in its preferred embodiment, is supplemented with yeast extract. Optionally, the medium of the disclosure, in its preferred embodiment, is supplemented with copper. Optionally, the medium of the disclosure, in its preferred embodiment, is supplemented with linoleic and linolenic acids.

The present disclosure provides a culture medium with natural inducer for biocomposites. In one aspect the present disclosure relates to a natural, low-cost sustainable medium containing an inducer of natural origin that enhances synthesis of biocomposites through cultivation of living cells. Specifically, the medium of the disclosure comprises as a source of macromolecules a natural substrate which incorporates a plant biomass with high copper content, a mixture of vitamins and trace minerals.

More specifically, the medium of the disclosure includes, in combination with the vegetable substrate, a natural inducer which is a vegetable vinegar obtained by the process of pyrolysis, rest and fractional distillation, until a totally reproducible product of refined pharmaceutical grade is obtained. The vinegar is composed of phenolic and polyphenolic compounds, organic acids, alcohols, cyclic ketones and other aromatic hydrocarbons, all present in trace amounts which are enhanced synergistically, and they avoid the use of high concentrations of any of them, which can be toxic. In particular, the toxicity evaluated for the natural inducer after the refining process was classified in category 5, or not classifiable, as assessed by MB Research Laboratories with Good Laboratory Practices (GLP), 1765 Wentz Road, PO Box 178, Spinnerstown, Pa. 18968.

The medium of the disclosure contains an inverse ratio of plant biomass and natural inducer. Particularly, in the disclosed medium the inducer is compatible with cell growth and is present in the initial composition of the medium, which makes it unnecessary to further supplement the medium with inducer. The disclosed medium is a functional culture medium which enables the optimization of the production of biocomposites. In a general aspect of the disclosure the medium is an indefinite functional culture medium with a natural biocomposites inducer and plant biomass incorporating a mixture of vitamins, high copper, trace minerals and the natural inducer.

This plant biomass incorporates high copper content not exceeding millimolar concentration, which is between 1 and 50% by total weight of the medium. The plant biomass incorporating high copper content of the disclosed medium comprises, in its most general aspect, comprises a mixture of vitamins selected from the group consisting of A, B1, B2, B12, D, E, folic acid, niacin and mixtures of the same in a specific proportion. This proportion of vitamins is in at least a micromolar concentration. The plant biomass incorporating high copper content of the disclosed medium comprises, in its most general aspect, a mixture of trace minerals selected from the group consisting of calcium, copper, phosphorus, magnesium, manganese, zinc, potassium and iron or compounds and mixtures thereof at a desired ratio. This proportion of trace minerals does not exceed millimolar concentration.

In another aspect of the disclosure the average plant biomass incorporating high copper content, trace vitamins and minerals may be, but is not limited to, fruit, nuts, grains, cereals, vegetables, tubers, legumes, cocoa, and derivatives, parts, and mixtures or combinations thereof. All these can be fresh or processed. In another aspect of the disclosed medium, the plant biomass incorporating high copper content, vitamins and trace minerals is selected from the group consisting of fruits, vegetables or cereals, or mixtures thereof. In another aspect of the disclosed medium, the plant biomass incorporating high copper content, vitamins and trace minerals is selected from the group consisting of banana fruit, banana, cacao, carob and its derivatives and mixtures thereof and nuts. In another aspect of the disclosed medium, the plant biomass incorporating high copper content, vitamins and trace minerals is selected from the vegetable group consisting of cucumber, cassava and potato tubers as derivatives and mixtures or parts thereof. In another aspect of the disclosed medium, the plant biomass incorporating high copper content, vitamins and trace minerals is selected from the group consisting of leguminous beans and lentils.

In another aspect of the disclosed medium, the plant biomass incorporating high copper content, vitamins and trace minerals is selected from the group consisting of corn grain, wheat, rice, oats, barley, corn, bran, their derivatives and mixtures or parts thereof. Optionally, the plant biomass that incorporates high copper content, vitamins and trace minerals may also contain bagasse, crop residue from all previous components, and sawdust of different woods, pastures or mixture thereof, fresh, processed or parts thereof. In its broadest aspect, the disclosed medium optionally comprises as carbon and energy source glucose and/or fructose syrup composed of maltose, maltotriose, glucose, oligosaccharides, fructose or dried material thereof in any combination or mixture.

In this general aspect of the disclosure the medium includes a natural inducer which is a grass vinegar refined to pharmaceutical grade. This natural inducer is between 1 and 50% by total weight of the medium. This natural inducer may come from any part of the plant, namely leaves, roots, stems, flowers, seeds or parts thereof. Optionally, in its broadest aspect, the means of the disclosure includes malt or yeast extract as an alternative source and/or additional nitrogen. Also optionally, the disclosed medium can be supplemented with copper. Further, optionally, the disclosed medium may contain long chain fatty acids, preferably linoleic and linolenic acids. In its broadest aspect the medium of the disclosure can be liquid, solid or semisolid.

The medium of the disclosure provides a natural environment, including biomass and inexpensive and nontoxic inducers of plant origin. The medium of the disclosure can be designed to produce multiple biocomposites. The composition of the disclosure medium provides optimal conditions for cell culture and production of biocomposites, without added toxic or synthetic substances or expensive supplements. Biocomposites obtained with the disclosed medium are achieved in a short time compared to conventional crops and in higher concentrations than those found in the prior art. The following examples relate to the preparation and application of the disclosure's culture medium.

These examples seek to illustrate the disclosure and therefore should not be construed in any way as limiting.

### 2.7.1. EXAMPLE 1

### Preparation of the Disclosed Medium

An indefinite functional medium for cell growth was designed and developed. Preparation of natural Inducer: Bambusoideae vinegar was obtained by a process of pyrolysis at high temperatures above 100° C. during the early stages of carbon production in the absence of oxygen. The steam produced by the pyrolysis process is condensed and collected. The product obtained is a light yellow or clear liquid, smelling of smoke, 1001-1008 g/cm3 specific gravity, pH between 3.4 and 3.7, and refined by distillation processes.

Preparing of pre-inocula and means of the disclosure: 4 cylindrical plugs were extracted from biomass previously planted in agar malt for planting of pre-inocula. The culture medium was prepared by mixing a refined vegetable vinegar inducer identified as pharmaceutical grade labeled R2 MVG-11M Lot No., container 1 of 19, incorporating plant biomass with high copper content, trace vitamins and minerals in a ratio inversely proportional to the inducer, glucose syrup and yeast extract. The medium was supplemented with copper compounds and linoleic and linolenic acids. Three Erlenmeyer flasks of 250 cc were prepared with 83 cc workload for each one. PH adjustment was performed with citrate phosphate buffer 0.1M citric acid and 0.2M sodium phosphate at 4.56. Sterilization of the components was performed by wet heat at 121° C., 103 psi for 15 minutes. Cylindrical blocks of the strain were added to each of the Erlenmeyer flasks and maintained at 30° C. in the incubator. This seed culture was subsequently used for the cultivation scale bioreactor. The medium for the instant disclosure was obtained for eukaryotic cell culture and kept at 4° C. or lyophilized.

Bioreactor scale culture. The culture medium is the same used for pre-inocula. Once components were homogenized and pH adjusted, it was sterilized at 121° C. at 718 Pa pressure for 15 minutes.

Bioreactor working conditions: Inoculum between 1 and 15%, temperature 25 to 35° C., pH between 3.5 and 5; stirring between 50 and 200 rpm; aeration between 1 to 3 L/min (±300 to 600% saturation of O2) volume of work=5000 cm3.

TABLE 1
Possible Mixtures

| Inducer | Plant Biomass |
|---|---|
| 1% | 2% |
| 2% | 1% |
| 2% | 4% |
| 4% | 2% |
| 4% | 8% |
| 8% | 4% |
| 8% | 16% |
| 16% | 8% |
| 16% | 24% |
| 24% | 16% |
| 24% | 48% |
| 48% | 24% |

### 2.7.2. EXAMPLE 2

### Production of Biocomposite 1

Cell culture in submerged fermentation in a synthetic medium and culture medium of the disclosure to induce production of Biocomposite 1. Production with the synthetic medium: Biocomposite 1 production was studied using a synthetic medium containing a copper inducer. The inducer was added on the fourth day after inoculating the medium. The activity of Biocomposite 1 was monitored for 20 days. Four concentrations of CuSO₄ were evaluated between 150-750 μM for the induction of Biocomposite 1 in submerged culture.

Production of Biocomposite 1 with the disclosed medium: The culture medium was prepared by mixing a refined vegetable vinegar inducer of pharmaceutical grade identified as R2 MVG-11M Lot No., container 1 of 19, with a combination of plant biomass in an inversely proportional relationship to the inducer, glucose syrup and yeast extract. The medium was supplemented with copper and linoleic and linolenic acids. Three erlenmeyers of 250 cc were prepared with a workload of 83 cc each. pH adjustment was performed with citrate phosphate buffer 0.1M citric acid and 0.2M sodium phosphate at 4.56. Sterilization of the components was performed by wet heat at 121° C., 103 psi for 15 minutes. Natural inducer concentration was maintained according to the initial composition of the disclosed medium.

Working conditions of the bioreactor: Inoculum between 1 and 15%; temperature between 25 and 35° C., pH between 3.5 and 5; stirring between 50 and 200 rpm; aeration between 1 to 3 L/min (±300 to 600% saturation of O₂) workload=5000 cm3.

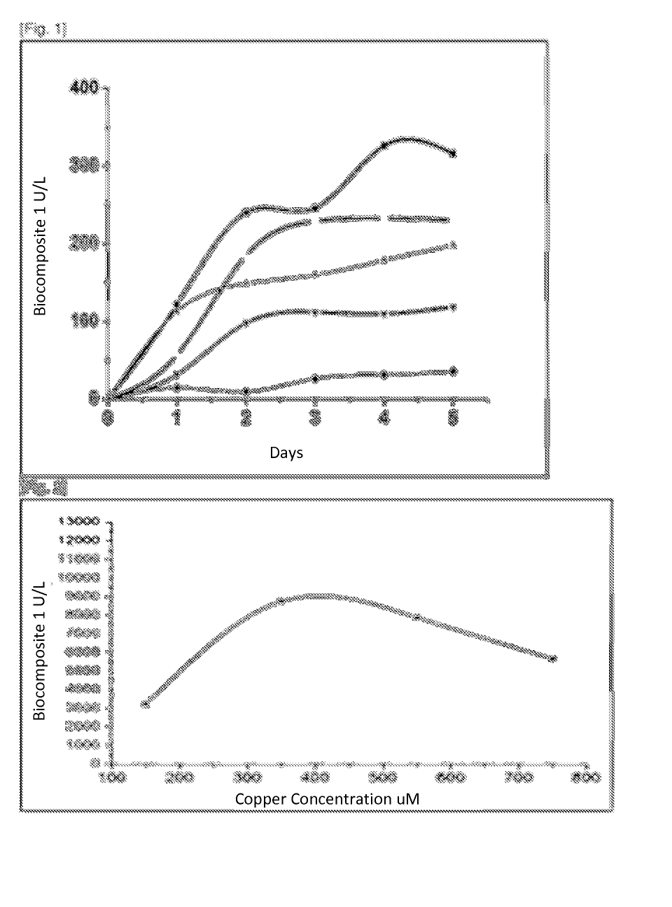

Cells in culture: four types of living cells were used, preserved in refrigeration and activated for use in vivo to evaluate the production of Biocomposite 1, denoted Sp1, Sp2, Sp3, Sp4 and Sp5, as shown in FIG. 1. For results of the production of Biocomposite 1, see FIG. 1. Production of Biocomposite 1 from cultivation of different eukaryotic cells -▪-Sp1, -□-Sp2. -▴-Sp3. -▾-Sp4. -♦-Sp5 in synthetic medium in the absence of inducers.

It was observed that in four cell cultures using the synthetic medium in the absence of any chemical inducer the maximum production of Biocomposite 1 found was 340 U/L, then it initiates a stationary phase and decreased metabolic activity to 0 U/L. Biocomposite 1: -▪-Sp1, the cell culture with highest production, was selected for induction with copper.

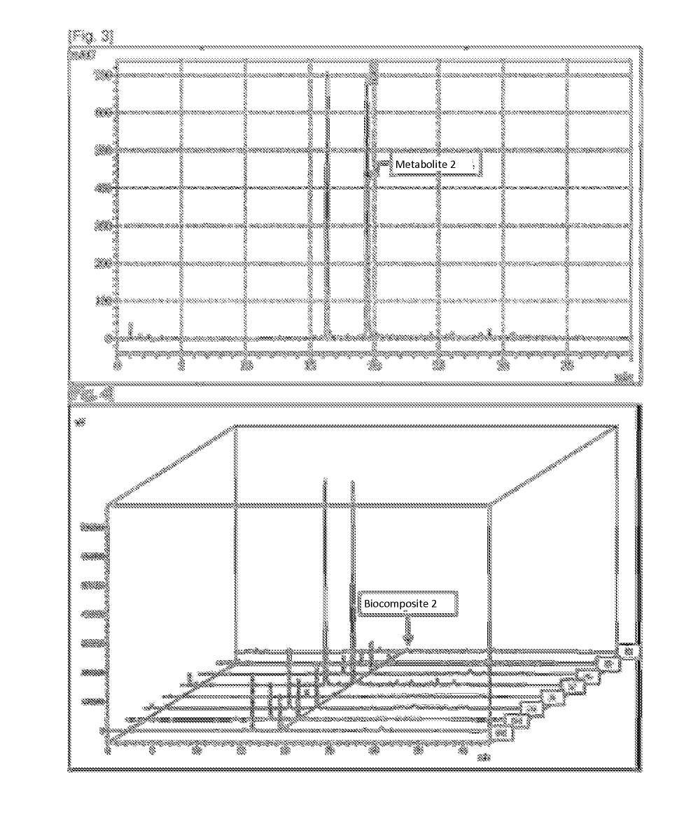

FIG. 2. Production of Biocomposite 1 from culture -. ▪-Sp1 in synthetic medium with copper inducer at concentrations between 100 and 750 uM. Data at 21 days of culture. The production of Biocomposite 1 -▪-Sp1 reached a maximum concentration of 11,650 U/L using a synthetic medium and a copper inducer. The average results for 3 replicates for production of Biocomposite 1 in the disclosed medium in submerged culture performed under the same conditions as the synthetic culture are presented in the following table.

Table 2. Determination of activity of Biocomposite 1 at 8 days of culture with the disclosed medium.

TABLE 2

| | Enzyme activity | | | |
|---|---|---|---|---|
| | Biocompound 1 | | | |
| | (U/L). | | | |
| Sample | Ri | R₂ | R₃ | Average |
| 1 | 18504 | 18701 | 21592 | 19599 |
| 2 | 24844 | 25544 | 28498 | 26295 |
| 3 | 26467 | 26746 | 28591 | 27268 |
| | | | | 24387 |

The production of Biocomposite 1 -▪-Sp1 reached a maximum concentration of 28,591 U/L using the disclosed medium.

Table 3. Comparison results for obtaining Biocomposite 1 in synthetic media and the disclosed media described above.

TABLE 3

| | Production High | | |
|---|---|---|---|
| Medium | Biocomposite 1 ng | | Time (Days) |
| Disclosed medium | 28089 | U/L | 8 days |
| Synthetic medium | 100-200 | U/L | 21 days |
| Synthetic medium | 8500 | U/L | 21 days |
| supplemented with | | | |
| inducers | | | |

In cell cultures grown in disclosed media higher concentrations of Biocomposite 1 were obtained with shorter culture.

### 2.7.3. EXAMPLE 3

### Production of Biocomposite 2

Cell culture in synthetic medium, natural medium and the disclosed medium for inducing production of Biocomposite 2 product from the secondary metabolism of the cultured cells. Preparation of cell cultures: Kirk synthetic medium was used as medium containing mainly carbon source, nitrogen, trace elements, vitamins and pH buffer.

As natural medium a solution composed of hydrolyzate agro residues was used. The culture medium of the present disclosure contains a natural vegetable vinegar inducer refined to pharmaceutical grade identified as R2, labeled MVG Lot No. 11M, container 1 of 19. A combination of vegetable biomass in an inversely proportional relation to the inducer, and also syrup, glucose; yeast extract and initial and linoleic and linolenic acids were added as a medium supplement. Once the components were homogenized and the pH adjusted the medium was sterilized at 121° C. at 718 Pa pressure for 15 minutes.

Working conditions of the bioreactor: Inoculum between 1 and 15%; temperature between 25 and 35° C., pH between 3.5 and 5; stirring between 50 and 200 rpm; aeration between 1 to 3 L/min (±300 to 600% saturation of O2) workload=5000 cm3.

Determination of Biocomposite 2

Extraction process: 10 g of biomass was dissolved in 250 mL of 70% methanol and left under stifling for 24 hours, then rotoevaporated; the aqueous phase was adjusted to pH 7 with 0.2M HCl and extracted with dichloromethane: ethyl acetate (90:10). The extract obtained was centrifuged at 4000 rpm for 5 minutes at 5° C., the organic phase removed and dried under nitrogen at 35° C.

Sample preparation for analysis: From the biomass obtained from the synthetic medium and the disclosed medium prepared as described in Example 1, samples of biomass were taken in dry triplicate of 50 grams, mixed with 70% ethanol (v/v) and stirred at room temperature for 5 days; then dried on a rotary evaporator under reduced pressure. Two samples were solubilized in different solvents, methanol, ethyl acetate and chloroform, extractions were performed and then rotoevaporated and dissolved in methanol for chromatographic analysis.

HPLC analysis: Analysis of the biomass by HPLC was carried out on a computer (High Resolution Liquid Chromatography Shimadzu brand), and diode array detector (SPD-M20A), and an autosampler controlled by an LC software solution. The analytical column used was RP-C18. The wavelength was at 270 nm. The process of elution of the samples was carried out with a flow rate of 1.0 ml/min at room temperature. Mobile phase A consisted of water and acetonitrile and Mobile phase B used isocratic elution. Injection volume was 20 ul. The concentration of Biocomposite 2 was determined in a calibration curve using a standard of Biocomposite 2.

Results of the production of Biocomposite 2. Biomass concentration in B2 medium obtained with the present disclosure: According to the calibration curve, the Biocomposite 2 present in the biomass is evaluated in an average concentration of 80.8220 ppm.

FIG. 3. Chromatogram of standard Biocomposite 2. FIG. 3 shows the peak of Biocomposite 2 in samples from cell cultures grown and appearing at a retention time of 20 minutes.

Biocomposite 2

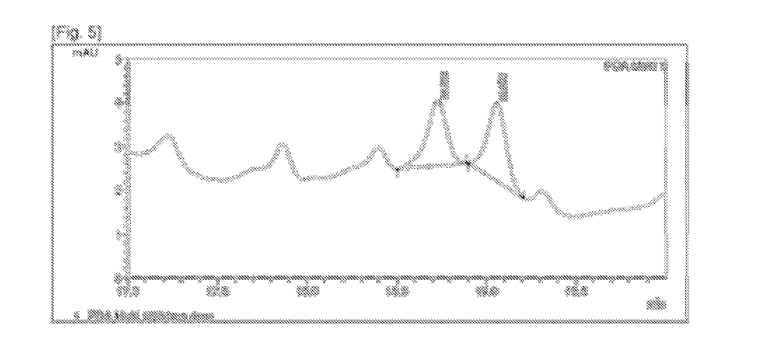

FIG. 4. Biocomposite 2 chromatograms obtained by different extraction methods. FIG. 4 shows differences in concentration of Biocomposite 2 according to the different extraction methods used; the chromatogram of the sample suggests that S7 is the best method of extraction and processing for the sample.

FIG. 5. Chromatogram with the peaks associated with the production of Biocomposite 2 from a synthetic medium. FIG. 5 shows that Biocomposite 2 is produced in the culture of the disclosed medium at a much higher concentration than that produced in a synthetic medium and a natural medium under the same culture conditions. Even in the natural environment, Biocomposite 2 was not detected.

Table 4. Comparison of production of Biocomposite 2 ppm produced with a synthetic, a natural and the disclosed media.

TABLE 4

| | Concentration | |
|---|---|---|
| Media | Biocomposite 2 | Cultivation time |
| Disclosed medium | 86.4145 mg/L | 10 days |
| Synthetic medium | 1.2 mg/L | 10 days |
| Natural medium | absence | — |

### 2.7.4. EXAMPLE 4

### Production of Biocomposite 3

Cell culture in a synthetic medium and in the disclosed medium for inducing production of Biocomposite 3. Preparation of cell cultures: Kirk synthetic medium was used as medium containing mainly carbon source, nitrogen, trace elements, vitamins and pH buffer. The culture medium used in this disclosure contains as natural inducer a refined vegetable vinegar of pharmaceutical grade identified as R2 and labeled MVG-11M Lot No., container 1 of 19. A combination of plant biomass in an inverse relationship to inducer, dextrose syrup; yeast extract and linoleic and linolenic acids was likewise added as medium supplement. Once the components were homogenized and the pH adjusted the medium was sterilized at 121° C. at 718 Pa pressure for 15 minutes.

Working conditions of the bioreactor: Inoculum between 1 and 15%; temperature between 25 and 35° C., pH between 3.5 and 5; stirring between 50 and 200 rpm; aeration between 1 and 3 L/min (±300 to 600% saturation of O2) workload=5000 cm3.

Results for the Production of Biocomposite 3

Table 5. Evaluation of Biocomposite 3production with the disclosed medium and a synthetic medium.

TABLE 5

| | | Biocomposite 3/5 Liter | |
|---|---|---|---|
| Medium | Time (Days) | Reactor | Percentage |
| Present disclosure | 7 days | Dry Weight: 480 gr | 9.6% |
| Synthetic | 7 days | Dry Weight: 331.02 gr | 6.6% |

The prior art has reported production of Biocomposite 3, using different carbon and nitrogen sources and indicated cultivation periods of minimum 45 days and up to 200 days in culture conditions comparable to those for the present example (E. Iclal Erke, 2009 and Mayszumi Et. al., 1993).

Proximate characterization of Biocomposite 3 in the inventive medium: Proximate analysis of Biocomposite 3 obtained in the inventive medium shows there is evidence of a protein content of 13%, fat 17%, and TDF (total dietary fiber) of 64%; likewise a total caloric value of 593.9 kcal/100 g as presented in the following tables.

Table 6. Proximate Analysis of Biocomposite 3 obtained with the disclosed medium.

TABLE 6

| Component | % |
|---|---|
| Protein | 13.0899 |
| Crude fat | 17.67 |
| FDT | 63.80 |

Biocomposite 3 produced with the means of the present disclosure has high protein and fiber content.

Table 7. Protein from Biocomposite 3 obtained by different media.

TABLE 7

| | Biocomposite 3 | |
|---|---|---|
| | (disclosed medium) | Biocomposite 3 (FES) |
| Protein | 13.1% | 7.3% |
| Cultivation time | 7 days | 30 days |

In the prior art it is reported that the protein content reached for Biocompo site 3 is close to 7% after 30 days of culture (Chiu Et. al., 2000). The Biocomposite 3 obtained with the disclosed medium has a caloric content of 13.1% in 7 days in culture.

### 2.7.5. EXAMPLE 5

### Production of Biocomposite 4

Culturing cells in a synthetic medium, a natural medium and the new medium disclosed herein for inducing production of Biocomposite 4. Preparation of cell cultures: Kirk synthetic medium was used as a medium containing mainly carbon source, nitrogen, trace elements, vitamins and pH buffer. As a natural medium a solution composed of hydrolyzate agro residues was used.

The culture medium used in this disclosure contains as natural inducer a refined vegetable vinegar at pharmaceutical grade identified as R2 and labeled MVG-11M Lot No., container 1 of 19. Plant biomass was also added to the medium, in an inversely proportional relation to the inducer, as were glucose syrup, extract of yeast and linoleic and linolenic acids as supplements. Once the components were homogenized and the pH adjusted the medium was sterilize to 121° C. at 718 Pa pressure for 15 minutes. Working conditions of the bioreactor: Inoculum between 1 and 15%; temperature between 25 and 35° C., pH between 3.5 and 5; stirring between 50 and 200 rpm; aeration between 1 and 3 L/min (±300 to 600% saturation of O2) workload=5000 cm3.

Results for the Production of Biocomposite 4

Table 8. Evaluation of Biocomposite 4 production from cell cultures with different media.

TABLE 8

| | | | | | | Concentration | |
|---|---|---|---|---|---|---|---|
| | ABSL | | | | | ppm (×Factor | |
| | λ = | Ab2 λ = | Ab3 λ = | | | of | % |
| Medium | 490 nm | 490 nm | 490 nm | Avg. | DS | dilution) | Biocomposite 4 |
| Disclosed | 0.361 | 0.268 | 0479 | 0.370 | 0.106 | 2723.611 | 4.36 |
| Medium | | | | | | | |
| Synthetic | 0.853 | 0.787 | 0.838 | 0.826 | 0.034 | 652 778 | 0.073 |
| Medium | | | | | | | |
| Natural | 0.520 | 0.498 | 0.546 | 0.521 | 0.024 | 398 778 | 0.250 |
| Medium | | | | | | | |

Table 8 shows the results of the concentration of Biocomposite 4 obtained with different culture media. It is noted that amounts produced with the medium, according the present disclosure under the same experimental conditions, are up to 7 times greater than those produced by the natural medium and up to 4 times greater than those produced in a synthetic medium. In the prior art it is known that the production of Biocomposite 4 is less than 1% at bioreactor scale for 30 days of culture (Joica Habijani Berovi and Marin, 2000).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A functional unlimited culture medium having a natural biocomposite inducer, the culture medium comprising:
a. a plant biomass incorporating at least 50 micromoles of copper and vitamins and trace minerals; and
b. a natural inducer comprising pharmaceutical grade stable vegetable ligneous vinegar.

2. The functional unlimited culture medium having a natural biocomposite inducer according to claim 1, wherein the natural inducer is present in an inverse ratio to the plant biomass content.

3. The functional unlimited culture medium having a natural biocomposite inducer according to claim 1, wherein the plant biomass is present in a proportion of at least 0.5%.

4. The functional unlimited culture medium having a natural biocomposite inducer according to claim 1, wherein the natural inducer is present in a proportion of at least 0.1%.

5. The functional unlimited culture medium having a natural biocomposite inducer according to claim 1, wherein the plant biomass comprises a mixture.

6. The functional unlimited culture medium having a natural biocomposite inducer according to claim 5, wherein the plant biomass is selected from the group consisting of a fruit, a vegetable, a cereal and mixtures thereof.

7. The functional unlimited culture medium having a natural biocomposite inducer according to claim 1, wherein the plant biomass is a mixture of grains.

8. The functional unlimited culture medium having a natural biocomposite inducer according to claim 1, wherein the plant biomass further comprises at least 10 micrograms of vitamins, up to a concentration of 1,000 milligrams vitamins per 100 grams of plant biomass.

9. The functional unlimited culture medium having a natural biocomposite inducer according to claim 8, wherein the plant biomass further comprises B complex group vitamins.

10. The functional unlimited culture medium having a natural biocomposite inducer according to claim 1, wherein the plant biomass further comprises trace minerals in a concentration of up to 10000 milligrams per 100 grams.

11. The functional unlimited culture medium having a natural biocomposite inducer according to claim 1, wherein the natural inducer comprises a vinegar from grasses.

12. The functional unlimited culture medium having a natural biocomposite inducer according to claim 1, wherein the natural inducer comprises a vinegar from Bambusoideae.

13. The functional unlimited culture medium having a natural biocomposite inducer according to claim 1, further comprising a glucose syrup, a fructose syrup or both a glucose syrup and a fructose syrup.

14. The functional unlimited culture medium having a natural biocomposite inducer according to claim 1, further comprising a malt extract, a yeast extract, or both a malt extract and a yeast extract.

15. The functional unlimited culture medium having a natural biocomposite inducer according to claim 1, further comprising copper in excess of 50 micromoles.

16. The functional unlimited culture medium having a natural biocomposite inducer according to claim 1, further comprising long chain fatty acids.

17. The functional unlimited culture medium having a natural biocomposite inducer according to claim 16, wherein the long chain fatty acids are selected from the group consisting of linoleic acid and linolenic acids.

18. The functional unlimited culture medium having a natural biocomposite inducer according to claim 1, wherein the functional unlimited culture medium is a liquid, a solid or a semisolid.

19. The functional unlimited culture medium having a natural biocomposite inducer according to claim 1, wherein the functional unlimited culture medium has been lyophilized.

20. The functional unlimited culture medium having a natural biocomposite inducer according to claim 1, wherein the plant biomass incorporates vitamins at an at least micromolar concentration.

21. A functional unlimited culture medium having a natural biocomposite inducer, comprising:
a. a plant biomass incorporating: at least 50 micromoles of copper; vitamins in a concentration of at least 10 micrograms per 100 grams; and trace minerals in a concentration of up to 10000 milligrams per 100 grams; and
b. a natural inducer comprising a pharmacological grade of stable vinegar from Bambusoideae.

22. The functional unlimited culture medium having a natural biocomposite inducer according to claim 21, wherein the medium is a liquid.

23. The functional unlimited culture medium having a natural biocomposite inducer according to claim 21, wherein the functional unlimited culture medium has been lyophilized.

24. The functional unlimited culture medium having a natural biocomposite inducer according to claim 21, further comprising a glucose syrup, a fructose syrup, or both a glucose and a fructose syrup.

25. The functional unlimited culture medium having a natural biocomposite inducer according to claim 21, further comprising a malt extract, a yeast extract, or both a malt extract and a yeast extract.

26. The functional unlimited culture medium having a natural biocomposite inducer according to claim 21, further comprising copper in excess of 50 micromoles.

27. The functional unlimited culture medium having a natural biocomposite inducer according to claim 21, further comprising long chain fatty acids.

28. The functional unlimited culture medium having a natural biocomposite inducer according to claim 27, wherein the long chain fatty acids are selected from the group consisting of linoleic acid and linolenic acids.

29. The functional unlimited culture medium having a natural biocomposite inducer according to claim 21, wherein the vitamins comprise at least one B-complex group vitamin.

30. A process for culture of microorganisms characterized by comprising a step of cell growth carried out in the medium according to claim 21.
